(19) United States
(12) Patent Application Publication
(10) Pub. No.: US 2011/0207949 A1
(43) Pub. Date: Aug. 25, 2011

(54) PROCESS FOR THE PREPARATION OF RAMELTEON

(75) Inventors: Manjunath Narayan Bhanu, Pune (IN); Chandrasekhar Sinha, Navi Mumbai (IN); Bhupesh Aher, Mumbai (IN); Amol Bandal, Raigad (IN); Atul Parab, Mumbai (IN)

(73) Assignee: WATSON PHARMA PRIVATE LIMITED, Andheri (E), Mumbai (IN)

(21) Appl. No.: 13/063,494

(22) PCT Filed: Nov. 12, 2009
(86) PCT No.: PCT/IB09/55038

§ 371 Date: Mar. 11, 2011

(30) Foreign Application Priority Data
Nov. 14, 2008 (IN) 2403/MUM/2008

(51) Int. Cl. C07D 307/93

(52) U.S. Cl. 549/458

(57) ABSTRACT

Disclosed herein is a process for resolving N-[2-(1,6,7,8-tetrahydro-2H-indeno [5,4-b] furan-8-yl)] ethylamine into its isomers using an optically active acid and a process for preparing ramelteon from the resolved isomer.

### FIELD OF THE INVENTION

The present invention relates to a process for the preparation of (S)-N-[2-(1,6,7,8-tetrahydro-2H-indeno [5,4-b] furan-8-yl]ethyl]propionamide, commonly known as ramelteon, in its pure isomeric form substantially free from its enantiomeric isomer.

### BACKGROUND OF INVENTION

Ramelteon (1) is a melatonin receptor agonist with both high affinity for melatonin MT₁ and MT₂ receptors and selectivity over the MT₃ receptor.

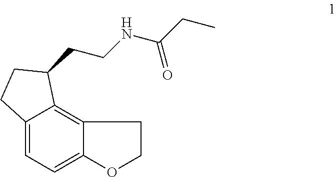

Ramelteon demonstrates full agonist activity in vitro in cells expressing human MT₁ or MT₂ receptors, and high selectivity for human MT₁ and MT₂ receptors compared to the MT₃ receptor.

Ramelteon has demonstrated efficacy in the treatment of insomnia characterized by difficulty with sleep onset. Approximately one in three American adults complains of some type of insomnia, and 20 million Americans suffer from chronic insomnia, which is characterized by difficulty falling asleep, difficulty staying asleep, or poor quality sleep, often leading to impairment of next-day functioning. Insomnia has been linked to a variety of health problems, including obesity, diabetes, hypertension, heart disease, and depression. Ramelteon has also been prescribed for long-term use in adults, provides a unique therapeutic mechanism of action for therapy of insomnia and represents a new treatment option.

U.S. Pat. No. 6,034,239 discloses the formation of chiral intermediates (S)-(−)-N-[2-(1,6,7,8,-tetrahydro-2H-indeno[5,4-b]furan-8-yl)ethylamine (sometimes referred to as compound S-2 or intermediate compound S-2) by the catalytic asymmetric hydrogenation of 2-(1,2,6,7,-tetrahydro-8H-indeno[5,4-b]furan-8-ylidene)ethylamine (compound 3 in the reaction scheme shown below) in the presence of a catalytic amount of BINAP-ruthenium complex in approximately 89% e.e. (enantiomeric excess). Following the catalytic reaction, the product is purified by preparing acid salts and acylated with propionyl chloride (compound 4 in the reaction scheme shown below) to obtain ramelteon (compound 1 in the reaction scheme shown below) in its pure (S) isomer form.

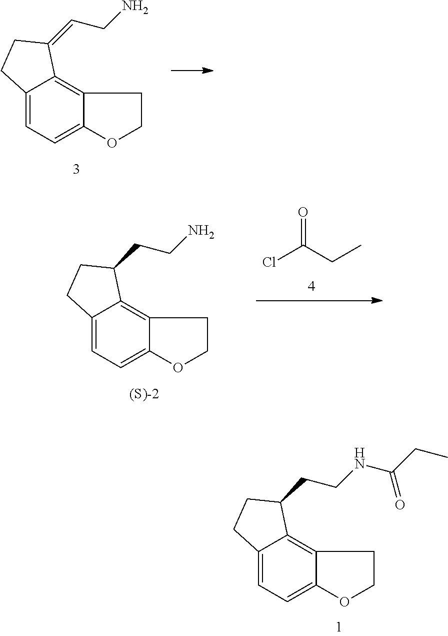

An alternate process for preparing ramelteon is disclosed in the Journal of Medicinal Chemistry, Vol. 45, pp. 4222-4239 (2002), wherein the exo double bond of intermediates (A) shown below was asymmetrically reduced using (S)-2,2′-bis-(diphenylphosphino)-1,1′-binaphthyl (binap)-Ru complex as the catalyst to obtain the enantiomerically pure compound (B). Compound (B) is subsequently converted to ramelteon (1) through the intermediate steps of Claisen condensation, ozonolysis and cyclization.

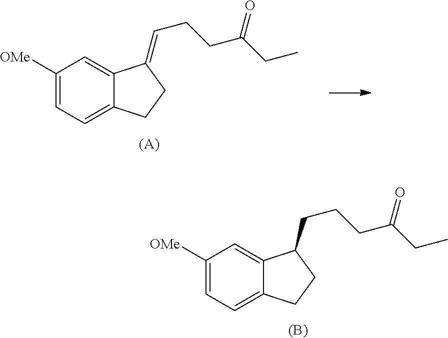

Both of the above processes uses expensive catalyst and give poor enantioselectivity. Additionally, these processes are expensive due to the need to perform multiple purifications steps in order to achieve an enantioselectivity of at least about 99% or greater of the desired isomer.

PCT Patent Publication No. WO 2008/062468 A2 discloses the following process for the preparation of ramelteon:

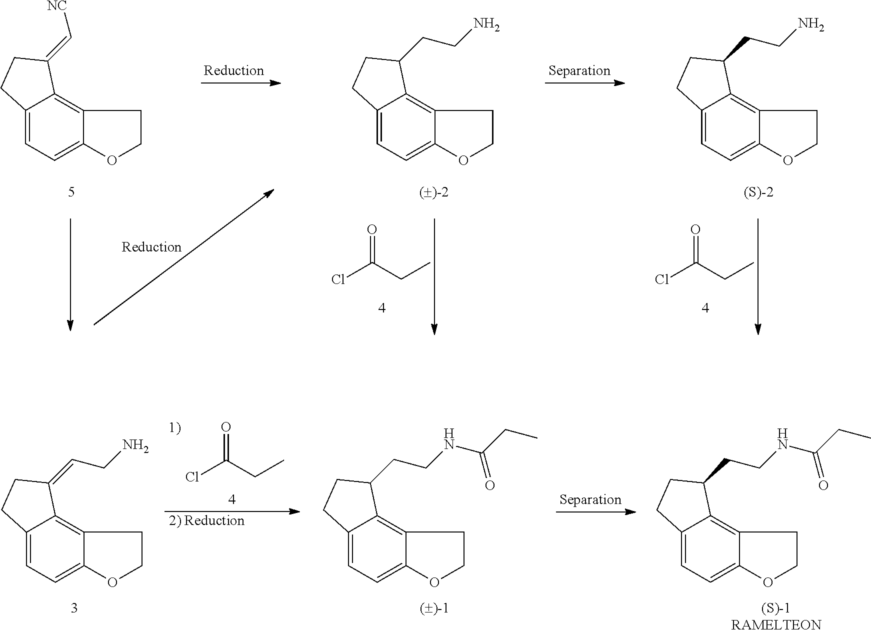

WO 2008/062468 teaches that separation of the enantiomers of intermediate (2) may be accomplished by: i) optical resolution of the racemic amine intermediate (2) by preparing acid salts with chirally pure acids; or ii) chromatographic techniques using chiral and/or achiral stationary phases for batch process, super critical or sub critical chromatography and/or continuous process chromatography. Although WO 2008/062468 mentions the possible use of optical resolution with chirally pure acids, there is no further teaching, discussion or disclosure of this method. WO 2008/062468 does, however, provide detailed descriptions of chromatographic methods for separating the isomers of intermediate compound (2). The disclosed chromatographic process suffers the following disadvantages:
Preparative chromatography is time consuming & expensive;
Highly sophisticated instrumentation required;
Not commercially feasible.

PCT Patent Publication No. WO 2008/106179 discloses a process for the preparation of ramelteon that involves the following reaction steps:

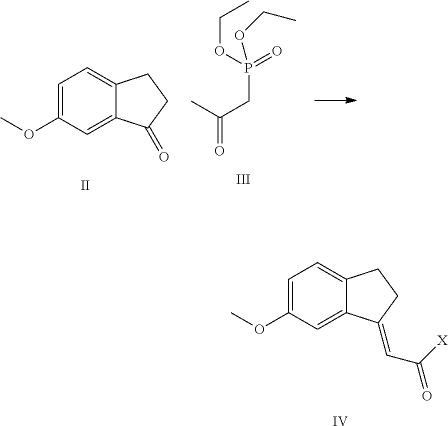

wherein X=O-alkyl or NH₂
and chiral reduction of the compound of formula IV in the presence of Ru-BINAP complex under hydrogen atmosphere in an organic solvent.

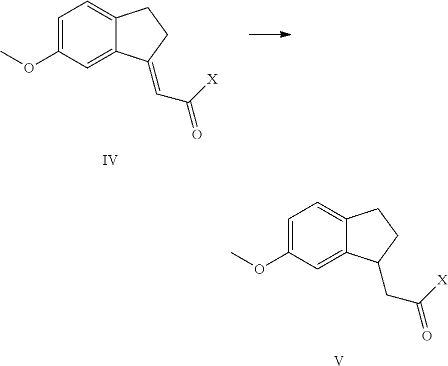

The process disclosed in WO 2008/106179 is similar to the process disclosed in U.S. Pat. No. 6,034,239 and the Journal of Medicinal Chemistry, Vol. 45 in that a Ru-BINAP complex is employed.

Resolution of racemic mixtures via reaction with optically active acids and the subsequent crystallization of the resulting salts is preferably employed when the chiral carbon of the racemic compound is an alpha carbon (i.e., one carbon removed) to the functional group forming the acid addition salt. As the distance between the chiral carbon of the racemic compound to the functional group of the racemic compound increases to beta (i.e., two carbon removed) & gamma (i.e., three carbon removed), the resolution of the diastereomeric salt becomes more difficult and not very useful.

Ramelteon has a chiral center at the gamma carbon, which makes the separation of the isomer with an optically active acid quite a daunting task. Similarly, N-[2-(1,6,7,8,-tetrahydro-2H-indeno [5,4-b]furan-8-yl)]ethylamine (compound 2), an intermediate useful in the production of ramelteon has a chiral center at the gamma carbon which would lead a skilled artisan to believe that optical resolution with an optically active acid could prove difficult.

Although a number of methods for preparing ramelteon are disclosed in the art, a need still exists to develop a process for preparing ramelteon in enantiomerically pure form which is cost effective, uses easily available reagents, is scalable with ease and overall commercially viable. This need and others is met by the present invention.

### SUMMARY OF THE PRESENT INVENTION

The present invention is a process for resolving N-[2-(1,6,7,8-tetrahydro-2H-indeno [5,4-b] furan-8-yl)]ethylamine (compound 2) into its isomers using an optically active acid to achieve high enantioselectivity of the desired isomer. The optically active acid is preferably a straight, branched or cyclic organic acid or a phenyl substituted organic acid.

The present invention further includes a process for the synthesis of ramelteon that comprises the step of separating N-[2-(1,6,7,8-tetrahydro-2H-indeno [5,4-b] furan-8-yl)]ethylamine (compound 2) into its isomers using an optically active acid to achieve high enantioselectivity of the desired isomer. This embodiment may further include the step of acylating the substantially pure enantiomer, (S)-N-[2-(1,6,7,8-tetrahydro-2H-indeno [5,4-b] furan-8-yl)]ethylamine (compound (S)-2) using a suitable acylating agent, such as propionyl chloride) to provide (S)-N-[2-(1,6,7,8-tetrahydro-2H-indeno[5,4-b]furan-8-yl]ethyl]propionamide (ramelteon or compound 1) substantially free of the (R)-isomer.

One embodiment of the present invention for the preparation of ramelteon is shown below in Scheme 1.

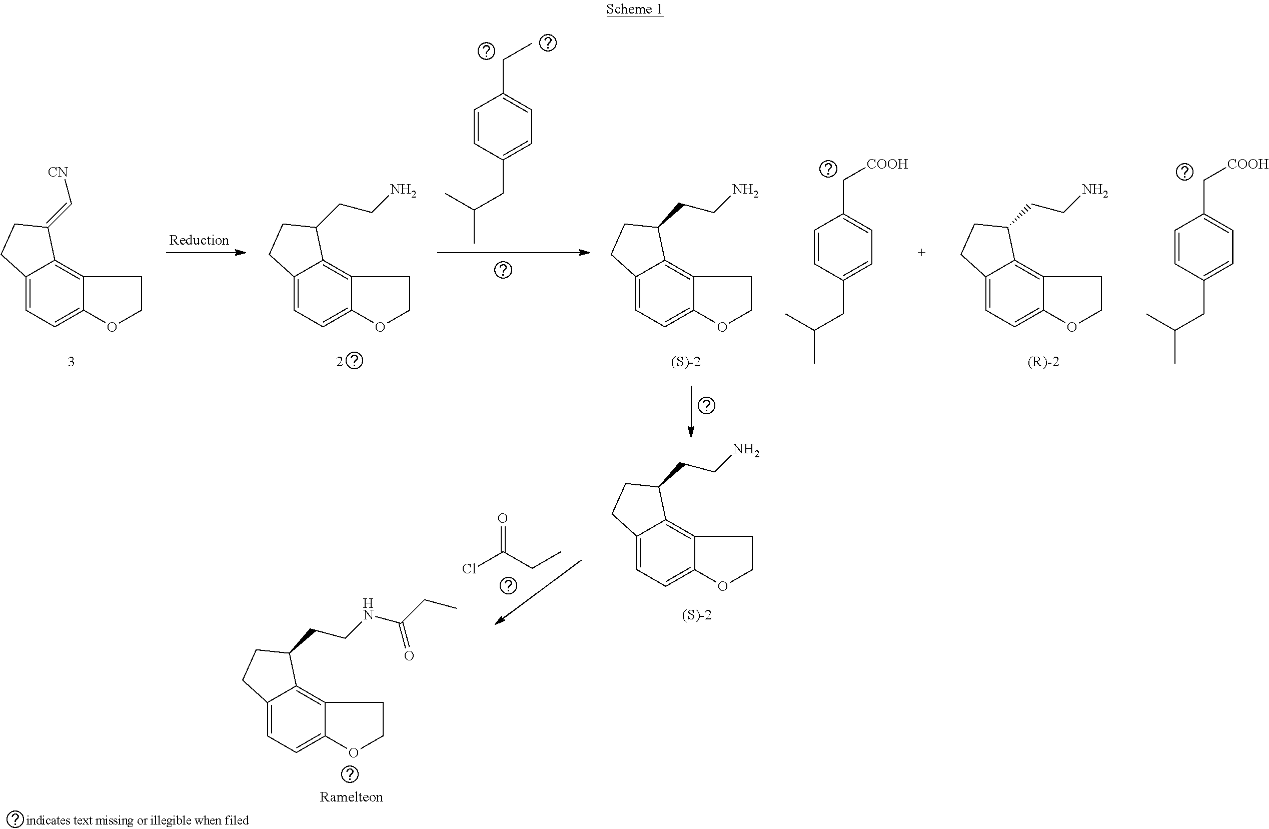

A further embodiment of the present invention includes a process for preparing ramelteon and (S)-N-[2-(1,6,7,8-tetrahydro-2H-indeno [5,4-b] furan-8-yl)]ethylamine (compound (S)-2) which does not employ any ruthenium complex or compounds.

A still further embodiment of the present invention includes a process for preparing ramelteon and (S)-N-[2-(1,6,7,8-tetrahydro-2H-indeno [5,4-b] furan-8-yl)]ethylamine (compound (S)-2) which does not employ any chromatographic purifications steps or procedures.

### DETAILED DESCRIPTION OF THE INVENTION

The present invention is a process for resolving N-[2-(1,6,7,8-tetrahydro-2H-indeno [5,4-b] furan-8-yl)]ethylamine (compound 2) into its isomers using an optically active acid to achieve high enantioselectivity of the desired isomer, preferably (S)-N-[2-(1,6,7,8-tetrahydro-2H-indeno [5,4-b] furan-8-yl)]ethylamine. The process comprises the step of:
i) reacting N-[2-(1,6,7,8-tetrahydro-2H-indeno [5,4-b] furan-8-yl)]ethylamine (compound 2) with an optically active acid to produce a diastereomeric salt of (S)-N-[2-(1,6,7,8-tetrahydro-2H-indeno [5,4-b] furan-8-yl)]ethylamine (compound (S)-2) and the optically active acid or a diastereomeric salt of (R)-N-2-(1,6,7,8-tetrahydro-2H-indeno [5,4-b] furan-8-yl) ethylamine (compound (R)-2) and the optically active acid;
ii) isolating (S)-N-[2-(1,6,7,8-tetrahydro-2H-indeno [5,4-b] furan-8-yl)]ethylamine (compound (S)-2) from the reaction mixture of step (i).

The N-[2-(1,6,7,8-tetrahydro-2H-indeno [5,4-b] furan-8-yl)]ethylamine (compound 2) employed in the present invention can be prepared by any means known in the industry such as those described in U.S. Pat. No. 6,034,239, WO 2008/062468 and WO 2008/106179. The N-[2-(1,6,7,8-tetrahydro-2H-indeno [5,4-b] furan-8-yl)]ethylamine (compound 2) can be reacted with the optically active acid by suspending or dissolving the N-[2-(1,6,7,8-tetrahydro-2H-indeno [5,4-b] furan-8-yl)]ethylamine (compound 2) and optically active acid in a solvent, preferably an organic solvent such as a C₁-C₆ alcohol and most preferably an organic solvent such as methanol, ethanol or isopropanol or mixtures thereof.

Examples of the optically active acid useful in the present invention include D-lactic acid, D-tartaric acid, D-malic acid, 1S-10-camphorsulfonic acid, S-hydratropic acid, (S)-2-methoxy phenyl acetic acid, (R)-2-methoxy-2-trifluoromethyl phenyl acetic acid, D-mandelic acid, di-P-anisoyl-D-tartaric acid, m-parachloro anilide, dibenzoyl-D-tartaric acid, S-(+)-1.1′-binaphthalene-2,2′-dihydrogen phosphate, S-2-(4-isobutylphenyl)propionic acid & mixtures thereof. The preferred optically active acids are straight, branched or cyclic organic acids such as D-lactic acid, D-tartaric acid, D-malic acid, 1S-10-camphorsulfonic acid or a phenyl substituted organic acid such as (S)-2-methoxy phenyl acetic acid, (R)-2-methoxy-2-trifluoromethyl phenyl acetic acid, D-mandelic acid, m-parachloro anilide, dibenzoyl-D-tartaric acid and S-2-(4-isobutylphenyl)propionic acid. The most preferred optically active acids are the aforementioned phenyl substituted organic acids or mixtures thereof.

The molar ratio of N-[2-(1,6,7,8-tetrahydro-2H-indeno [5,4-b] furan-8-yl)]ethylamine (compound 2) to optically active acid can range from about 1:0.5 to about 1:5, preferably about 1:0.75 to about 1:3 and most preferably about 1:0.9 to about 1:1.3.

The isolation of (S)-N-2-(1,6,7,8-tetrahydro-2H-indeno [5,4-b] furan-8-yl)ethylamine (compound (S)-2) from the reaction mixture N-[2-(1,6,7,8-tetrahydro-2H-indeno [5,4-b] furan-8-yl)]ethylamine (compound 2) and the optically active acid can be accomplished by any means commonly used in the chemical arts such as solvent extraction or crystallization.

One embodiment of the present invention comprises reacting N-[2-(1,6,7,8-tetrahydro-2H-indeno [5,4-b] furan-8-yl)]ethylamine (compound 2) with an optically active acid, preferably a phenyl substituted organic acid such as S-2-(4-isobutylphenyl)propionic acid, to produce a diastereomeric salt of (S)-N-[2-(1,6,7,8-tetrahydro-2H-indeno [5,4-b] furan-8-yl)]ethylamine (compound (S)-2) and the optically active acid. The salt of compound (S)-2 is isolated from the reaction mixture, preferably by precipitation, and then purified by conventional techniques such as recrystallization to obtain a salt of compound (S)-2 having a chiral purity of greater than 98% enantioselectivity, preferably greater than 98.5% enantioselectivity and most preferably greater than 99.0% enantioselectivity.

The purified salt is then converted to the free base form of (S)-N-[2-(1,6,7,8-tetrahydro-2H-indeno [5,4-b] furan-8-yl)]ethylamine (compound (S)-2) by conventional techniques. One embodiment of this aspect of the invention obtains the free base by suspending the purified salt of (S)-N-[2-(1,6,7,8-tetrahydro-2H-indeno [5,4-b] furan-8-yl)]ethylamine (compound (S)-2) and the optically active acid in an appropriate solvent such as water and adjusting the pH of the aqueous suspension to about 8-13, preferably 9-12 and most preferably about 10-12. The pH may be adjusted by adding an appropriate base such as aqueous sodium hydroxide to the aqueous suspension. After the pH has been adjusted, the free base form of (S)-N-[2-(1,6,7,8-tetrahydro-2H-indeno [5,4-b] furan-8-yl)]ethylamine (compound (S)-2) is isolated from the reaction mass. The free base form of compound (S)-2 can be isolated by any conventional means known in the chemical arts.

One embodiment of the present invention isolates the free base form of (S)-N-[2-(1,6,7,8-tetrahydro-2H-indeno [5,4-b] furan-8-yl)]ethylamine (compound (S)-2) from the basic aqueous suspension by an extraction with an appropriate organic solvent, preferably an aprotic solvent and most preferably a halogenated organic solvent such as dichloromethane.

Once the free base form of (S)-N-[2-(1,6,7,8-tetrahydro-2H-indeno [5,4-b] furan-8-yl)]ethylamine (compound (S)-2) is isolated from the basic aqueous suspension, it may be washed, dried and/or further purified to produce (S)-N-[2-(1,6,7,8-tetrahydro-2H-indeno [5,4-b] furan-8-yl)]ethylamine (compound (S)-2) with a chiral purity of greater than 98% enantioselectivity, preferably greater than 98.5% enantioselectivity and most preferably greater than 99.0% enantioselectivity.

A further embodiment of the present invention comprises the additional step of converting the (S)-N-[2-(1,6,7,8-tetrahydro-2H-indeno [5,4-b] furan-8-yl)]ethylamine (compound (S)-2) with a chiral purity of greater than 98% enantioselectivity, preferably greater than 98.5% enantioselectivity and most preferably greater than 99.0% enantioselectivity, into ramelteon.

The (S)-N-[2-(1,6,7,8-tetrahydro-2H-indeno[5,4-b] furan-8-yl)] ethylamine (compound (S)-2) can be converted to ramelteon by acylating the (S)-N-[2-(1,6,7,8-tetrahydro-2H-indeno[5,4-b]furan-8-yl)]ethylamine (compound (S)-2) using a suitable acylating agent, such as propionyl chloride, to produce ramelteon. A number of processes for acylating (S)-N-[2-(1,6,7,8-tetrahydro-2H-indeno[5,4-b]furan-8-yl)]ethylamine (compound (S)-2) to produce ramelteon are described in U.S. Pat. No. 6,034,239, Japanese Patent Publication No. 11080106, WO 2008/062468 and WO 2008/106179.

One aspect of the present invention for the preparing ramelteon comprises the steps of:
i) dissolving the (S)-N-[2-(1,6,7,8-tetrahydro-2H-indeno[5,4-b] furan-8-yl)] ethylamine (compound (S)-2) prepared by the above described resolution process with an optically active organic acid in a suitable organic solvent, preferably a polar aprotic solvent such as dimethylsulfoxide, dimethylformamide and tetrahydrofuran and more preferably a halogenated organic solvent such as dichloromethane;
ii) adding an acyling agent, preferably propionyl chloride, to solution of step (i); and
iii) isolating the ramelteon from the reaction mixture of step (ii).

The molar amount of acylating agent, i.e., propionyl chloride, employed in the above process should be equivalent or slightly in excess of the molar amount of (S)-N-[2-(1,6,7,8-tetrahydro-2H-indeno[5,4-b]furan-8-yl)]ethylamine (compound (S)-2). Preferably the molar ratio of acylating agent to compound (S)-2 should be about 1:1 to about 2:1, preferably about 1:1 to about 1.5:1. The acylation reaction may occur under ambient conditions, i.e., room temperature and normal atmospheric pressure.

It is also desirable to include a base in the solution of step (i) and/or the reaction mixture of step (ii) to react with the acid formed during the acylation reaction. Examples of some of the bases that can be added during the process are tertiary amines such as triethyl amine and diisopropyl ethylamine. The amount of the base added to the above process should be molar equivalent to the amount of acylating agent added during step (ii).

Once the acylation reaction is completed, the ramelteon may be isolated from the reaction mixture by any conventional methods known in the chemical arts. One embodiment of the present invention employs a solvent extraction wherein water is added to the reaction mixture and the organic solvent of step (i), which contains the ramelteon, is separated from the aqueous layer. The organic solvent is then removed to obtain the ramelteon. The resulting ramelteon may be purified to obtain a final product with a chiral purity of greater than 98% enantioselectivity, preferably greater than 98.5% enantioselectivity and most preferably greater than 99.0% enantioselectivity.

### EXAMPLES

### Example 1

### Preparation of (S)-N-2-(1,6,7,8-tetrahydro-2H-indeno [5,4-b] furan-8-yl) ethylamine (Compound (S)-2)

A solution of N-[2-(1,6,7,8-tetrahydro-2H-indeno [5,4-b] furan-8-yl)ethylamine (45 g; 0.22 mol) in methanol (225 ml) is added to a solution of S-(+)-2-(4-isobutylphenyl)propionic acid (41 g; 0.20 mol) in methanol (205 ml) at 25-30° C. The reaction mixture is concentrated to dryness under reduced pressure. The crude salt precipitated is recrystallized in methanol to give a diastereomeric salt of (S)-N-2-(1,6,7,8-tetrahydro-2H-indeno [5,4-b] furan-8-yl) ethylamine with (S)-(+)-2-(4-isobutylphenyl) propionic acid having a chiral purity of greater than 90% enantioselectivity.

The product obtained is recrystallized from methanol to give the pure salt having chiral purity of 99% or greater enantioselectivity.

The purified salt is suspended in water and the pH of the suspension is adjusted to 11-12 using aqueous sodium hydroxide. The reaction mixture is extracted with dichloromethane, washed with water and evaporated to give the pure (S)-N-[2-(1,6,7,8-tetrahydro-2H-indeno [5,4-b] furan-8-yl)]ethylamine (compound (S)-2), substantially free from its (R) isomer.

### Example 2

### Preparation of (S)-N-[2-(1,6,7,8-tetrahydro-2H-indeno [5,4-b] furan-8-yl)ethyl] propionamide (ramelteon)

Triethyl amine (15.15 g, 0.15 mol) and propionyl chloride (13.66 g, 0.15 mol) were added to a solution of S-[2-(1,6,7,8-tetrahydro-2H-indeno [5,4-b]furan-8-yl)]ethylamine (25 g, 0.12 mol) (compound (S)-2) (prepared in Example 1) in dichloromethane and stirred at room temperature for 2 hours. 75 mL water was added to the reaction mixture, and the layers were separated. The dichloromethane layer was concentrated under reduced pressure and purified from a mixture of acetone and hexane to give (S)-N-[2-(1,6,7,8-tetrahydro-2H-indeno [5,4-b] furan-8-yl) ethyl] propionamide (compound 1) having a chiral purity of 99% or greater enantioselectivity.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A process for resolving N-[2-(1,6,7,8-tetrahydro-2H-indeno [5,4-b] furan-8-yl)] ethylamine comprising the step of:
i) reacting N-[2-(1,6,7,8-tetrahydro-2H-indeno [5,4-b] furan-8-yl)]ethylamine with an optically active acid to produce a salt of (S)-N-2-(1,6,7,8-tetrahydro-2H-indeno [5,4-b] furan-8-yl) ethylamine and the optically active acid or a salt of (R)-N-2-(1,6,7,8-tetrahydro-2H-indeno [5,4-b] furan-8-yl) ethylamine and the optically active acid;
ii) isolating (S)-N-2-(1,6,7,8-tetrahydro-2H-indeno [5,4-b] furan-8-yl) ethylamine from the reaction mixture of step (i) wherein the process does not employ any ruthenium complex or compounds.

2. The process as described in claim 1 wherein the optically active acid is a straight, branched or cyclic organic acid.

3. The process as described in claim 2 wherein the optically active acid is selected from the group consisting of D-lactic acid, D-tartaric acid, D-malic acid, 1S-10-camphorsulfonic acid and combinations thereof.

4. The process as described in claim 1 wherein the optically active acid is a phenyl substituted organic acid.

5. The process as describe in claim 4 wherein the optically active acid is selected from the group consisting of (S)-2-methoxy phenyl acetic acid, (R)-2-methoxy-2-trifluoromethyl phenyl acetic acid, D-mandelic acid, m-parachloro anilide, dibenzoyl-D-tartaric acid, 5-2-(4-isobutylphenyl)propionic acid and combinations thereof.

6. The process as described in claim 5 wherein the optically active acid is S-2-(4-isobutylphenyl)propionic acid.

7. The process of claim 1 further comprising the step of converting the (S)-N-2-(1,6,7,8-tetrahydro-2H-indeno [5,4-b] furan-8-yl) ethylamine from step (ii) into ramelteon.

8. The process of claim 7 wherein the converting step comprises:
a) dissolving the (S)-N-[2-(1,6,7,8-tetrahydro-2H-indeno[5,4-b]furan-8-yl)]ethylamine in an organic solvent;
b) adding an acyling agent to the solution of step (a); and
c) isolating the ramelteon from the reaction mixture of step (b).

9. The process of claim 8 wherein the organic solvent of step (a) is a polar aprotic solvent.

10. The process of claim 8 wherein the organic solvent is selected from the group consisting of dimethylsulfoxide, dimethylformamide, tetrahydrofuran and mixtures thereof.

11. The process of claim 8 wherein the organic solvent of step (a) is a halogenated organic solvent.

12. The process of claim 8 wherein the organic solvent is dichloromethane.

13. Ramelteon produced according to the process of claim 7.

14. Ramelteon produced according to the process of claim 8.

15. The process according to claim 1 which does not employ any chromatographic purifications steps or procedures.
